(19) United States
(12) Patent Application Publication
(10) Pub. No.: US 2023/0314292 A1
(43) Pub. Date: Oct. 5, 2023

(54) DILUTION SOLUTION FOR MEASURING FREE STEROIDS IN BLOOD AND METHOD FOR MEASURING STEROID USING THE SAME

(71) Applicant: KOREA INSTITUTE OF SCIENCE AND TECHNOLOGY, Seoul (KR)

(72) Inventors: Man Ho CHOI, Seoul (KR); Jong Sung NOH, Seoul (KR); Seuk Min RYU, Seoul (KR)

(73) Assignee: KOREA INSTITUTE OF SCIENCE AND TECHNOLOGY, Seoul (KR)

(21) Appl. No.: 18/078,328

(22) Filed: Dec. 9, 2022

(30) Foreign Application Priority Data
Apr. 5, 2022 (KR) 10-2022-0042183

(51) Int. Cl. G01N 1/38; G01N 33/74; C01G 9/06; C01D 1/04; C01D 7/00

(52) U.S. Cl. CPC G01N 1/38; G01N 33/743; C01G 9/06; C01D 1/04; C01D 7/00

(57) ABSTRACT

The present invention relates to a diluted solution for measuring free steroids in blood and a method for measuring free steroids using the same, and more particularly, to a method for preparing a diluted blood sample for measuring free steroids in blood and a composition for blood sample dilution solution.

In the method for preparing a diluted blood sample for measuring free steroids in blood of the present invention, it was confirmed that physiologically active free cortisol could be evaluated with the cortisol enzyme immunoreaction detection kit without additional sample pretreatment by removing cortisol which is present by binding to excess albumin and cortisol-specific corticosteroid-binding globulin (CBG) contained in serum using a protein precipitation and neutralizing agent. In addition, by selectively removing the protein in the blood sample, it is possible to measure low molecular weight compounds, including steroids, which are strongly bound to proteins in the blood, such as cortisol, in free form.

Therefore, the method of the present invention can be applied simply and effectively to medical sites requiring blood tests for disease diagnosis, monitoring of free cortisol before and after surgery, and emergency patients.

This application claims the benefit under 35 USC § 119(a) of Korean Patent Application No. 10-2022-0042183, filed on Apr. 5, 2022 in the Korean Intellectual Property Office, the entire disclosure of which is incorporated herein by reference for all purposes.

### TECHNICAL FIELD

The present invention relates to a diluted solution for measuring free steroids in blood and a method for measuring free steroids using the same, and more particularly, to a method for preparing a diluted blood sample for measuring free steroids in blood and a composition for blood sample dilution solution.

### BACKGROUND ART

Steroid is a signal transduction hormone in the body and is a general term for substances having the basic structure of steroids or the similar biochemical functions, includes progestogen, corticoid, androgen, estrogen produced from sterol through metabolic processes in the body, and bile acid and vitamin D, which are biosynthesized from cholesterol, as well as steroid-based drugs such as dexamethasone as synthetic substances.

Cortisol, one of the corticosteroids, is a representative hormone of glucocorticoids produced and secreted by the adrenal cortex. It maintains homeostasis in the body by the regulatory functions of the hypothalamus and pituitary, and is involved in various metabolic physiology including response to stress and glucose metabolism. Thus, when cortisol is excessively produced or deficient in the body due to an adrenal or pituitary dysfunction, endocrine diseases such as Cushing's syndrome or Addison's disease develop.

Therefore, it is essential to monitor a crisis situation of hypocortisolism that can occur in diagnosing various adrenal and pituitary-related diseases, in severe emergency patients, acute hyponatremia patients with impaired consciousness, and septic shock due to infection by the measurement of cortisol in the blood. In addition, the monitoring of the cortisol concentration in the body before and after surgery for resection of the adrenal gland and pituitary gland, and surgical operation such as rheumatoid arthritis is very important for maintaining the health and life of patients due to adrenal insufficiency and adrenal crisis.

About 90-95% of cortisol in the blood is bound to corticosteroid-binding globulin (CBG) and albumin, and less than 5% exists as free cortisol, which exhibits physiological activity. However, antibody-based immunoassays used in clinical practice measure not only free cortisol but also protein-bound cortisol, that is, total cortisol. The importance of free cortisol analysis is being emphasized because it may indicate erroneous analysis results (N. Eng. J. Med., 350: 1629-1638, 2004). In the case of free cortisol, it can be measured by converting the amount of total cortisol and CBG measured in the blood to the free cortisol index (FCI), or through a complex sample pretreatment process such as high-efficiency filtration or equilibrium dialysis (Clin. Chem. Lab. Med., 45(4): 5210525, 2007), it is not performed in an actual diagnostic laboratory due to the inaccuracy of the test results and the time required for sample preparation and it is mainly analyzed by mass spectrometry in a separate testing agency.

In the present invention, it was attempted to develop a sample pretreatment technology capable of measuring free cortisol more simply and accurately in the medical field by simply removing the protein based on simple dilution of the blood.

### DISCLOSURE

### Technical Problem

The present inventors can selectively measure free cortisol by precipitating proteins in serum separated from blood using a protein precipitation solution, and removing cortisol bound to albumin and CBG by adding an alkaline neutralizing solution to inactivate the remaining precipitation solution, and thus, a diluted serum was obtained (FIG. 1). It was confirmed that the serum diluted by the method of the present invention can evaluate the free cortisol concentration instead of the total cortisol by the enzyme immunization method without additional pretreatment steps, and as a free steroid with clinical significance, it was also confirmed that free testosterone and free cholesterol can be selectively separated and detected, and thus, the present invention was completed.

Accordingly, an object of the present invention is to provide a method for preparing a diluted blood sample for measuring free steroids in blood.

Another object of the present invention is to provide a composition for a blood sample dilution solution for measuring free steroids in blood. Another object of the present invention is to provide a method for preparing the diluted blood sample or a method for measuring free steroids in blood using the present composition.

### Technical Solution

In order to achieve the above object, the present invention provides a blood sample dilution solution composition for measurement of free steroids in blood, wherein the composition comprises a zinc sulfate (ZnSO₄) solution and an alkaline neutralizing solution.

In a preferred embodiment of the present invention, the steroid may be any steroid present in a free form in the blood.

In another embodiment of the present invention, the steroid may be cortisol.

In another embodiment of the present invention, the blood may be whole blood, serum or plasma.

In another embodiment of the present invention, proteins in the blood are precipitated by the zinc sulfate solution.

In another embodiment of the present invention, 1 to 2 mL of 1 to 125 mM zinc sulfate solution is added per 1 mL of the blood sample.

In another embodiment of the present invention, the alkaline neutralizing solution has the same molar concentration and addition amount as the zinc sulfate solution.

In another embodiment of the present invention, the alkaline neutralizing solution inactivates the remaining zinc ions when the protein is precipitated with the zinc sulfate solution.

In another embodiment of the present invention, the alkaline neutralizing solution is any one selected from the group consisting of sodium hydroxide, potassium hydroxide, sodium carbonate and.

In another embodiment of the present invention, the composition further comprises a sodium acetate solution for pH adjustment.

In another embodiment of the present invention, the pH may be adjusted to be 6.2 to 7.5.

In another embodiment of the present invention, the composition is for measuring free steroids in blood by using an enzyme immunoreaction method or mass spectrometry.

In order to achieve the other objects described above, the present invention provides a method for preparing a diluted blood sample for measuring free steroids in blood, comprising: (a) adding zinc sulfate (ZnSO₄) solution to a blood sample to precipitate the protein; and (b) adding an alkaline neutralizing solution for zinc ion inactivation to the solution in which the protein is precipitated, and then centrifuging to obtain a supernatant.

In order to achieve another object described above, the present invention provides a method for measuring free steroids in blood comprising: measuring a concentration of free steroids in a sample prepared by the method for preparing a diluted blood sample for measuring free steroids in blood according to the present invention.

### Advantageous Effects

A blood sample dilution solution composition for measurement of free steroids in blood of the present invention, it was confirmed that physiologically active free cortisol could be evaluated with the cortisol enzyme immunoreaction detection kit without additional sample pretreatment by removing cortisol which is present by binding to excess albumin and cortisol-specific corticosteroid-binding globulin (CBG) contained in serum using a protein precipitation and neutralizing agent. In addition, by selectively removing the protein in the blood sample, it is possible to measure low molecular weight compounds, including steroids, which are strongly bound to proteins in the blood, such as cortisol, in free form.

Therefore, a blood sample dilution solution of the present invention can be applied simply and effectively to medical sites requiring blood tests for disease diagnosis, monitoring of free cortisol before and after surgery, and emergency patients.

### DESCRIPTION OF DRAWINGS

FIG. 1 is a schematic diagram showing a method for preparing a diluted blood sample for measuring free steroids in blood according to the present invention.

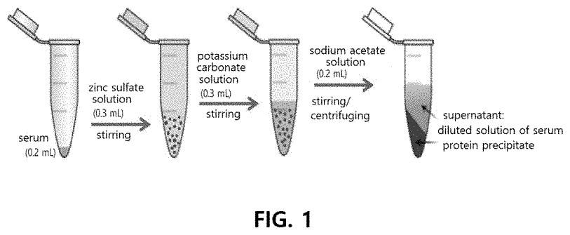

FIG. 2 is a photograph of precipitating proteins in a blood sample by sequentially treating the serum (A) and whole blood (B) samples with a zinc sulfate solution and a potassium carbonate solution.

### Mode of the Invention

Hereinafter, the present invention will be described in detail.

A monitoring of cortisol concentration is essential in emergency situations such as unconscious emergency patients or inflammatory sepsis. Since the cortisol immunoassay kit evaluates both cortisol bound to a protein present in blood and free cortisol exhibiting physiological activity, there is a problem in that the efficiency is lowered. In the case of measuring free cortisol using immunoassay and mass spectrometry combined with organic solvent extraction, it is not suitable for real-time monitoring in mass spectrometry due to complicated sample preparation and the organic solvent is a problem that it cannot be utilized in an immunoassay-based kit.

In the present invention, in order to measure free cortisol, a blood sample dilution solution that does not interfere with the antigen-antibody reaction of the immunoassay-based kit while removing the protein by simply diluting the blood was developed.

The blood sample dilution solution of the present invention comprises a zinc sulfate solution as a protein precipitation solution, and a potassium carbonate solution as a neutralizing solution for neutralizing protein-bound inorganic ions remaining in the solution after protein precipitation. Rapid detection of compounds such as testosterone and cholesterol is possible.

Accordingly, the present invention in this respect, relates to a blood sample dilution solution composition for measurement of free steroids in blood, wherein the composition comprises a zinc sulfate (ZnSO₄) solution and an alkaline neutralizing solution.

In another aspect, the present invention relates to a method for preparing a diluted blood sample for measuring free steroids in blood, comprising: (a) adding zinc sulfate (ZnSO₄) solution to a blood sample to precipitate the protein; and (b) adding an alkaline neutralizing solution for zinc ion inactivation to the solution in which the protein is precipitated, and then centrifuging to obtain a supernatant.

In the present invention, the steroid may be a steroid hormone that exists in a free form, preferably cortisol.

In the present invention, the blood may be whole blood, serum or plasma.

In the present invention, 1 to 2 mL of 1 to 125 mM zinc sulfate solution may be added per 1 mL of blood sample in step (a).

The concentration of zinc sulfate used as the protein precipitation solution can be used in proportion to the amount of blood and serum or plasma separated from blood used in clinical practice, and 1 to 2 mL of 1 to 125 mM zinc sulfate solution per 1 mL of blood sample can be added. The zinc sulfate solution may preferably have a concentration of 1 to 10 mM, and more preferably, may have a concentration of 2 to 5 mM.

In the present invention, the alkaline neutralization solution may be any one selected from the group consisting of sodium hydroxide, potassium hydroxide, sodium carbonate and potassium carbonate solution, preferably potassium carbonate solution.

In the present invention, the alkaline neutralization solution in step (b) may be added in the same molar concentration and amount as the zinc sulfate solution.

In the present invention, in order to neutralize the zinc ions remaining in the mixed solution after protein precipitation, an alkaline neutralizing solution can be added, and the alkaline neutralizing solution can react with zinc ions to form zincate. The alkaline neutralization solution can be used in the same equivalent ratio as the zinc sulfate solution in step (a) in order to remove zinc ions, but the concentration and amount of addition may be adjusted according to the purpose without being limited thereto.

In the present invention, the protein was precipitated by adding the zinc sulfate solution (protein precipitation solution), and then the neutralization solution can be added immediately. However, if necessary, the zinc sulfate solution can be mixed with serum, the mixture can be centrifuged, and then the neutralization solution can be added to the supernatant.

Since there is no significant difference in the extraction efficiency of the actual free cortisol between the two methods, it can be carried out in consideration of the simplicity of the process. In addition, in case that the sample pretreatment is performed after pre-mixing the protein precipitation solution and the neutralization solution, the recovery rate of free steroids can be lowered.

In the present invention, after zinc ion inactivation in step (b), sodium acetate solution may be additionally added to adjust the pH, and the pH may be adjusted to be 6.2 to 7.5 for steroid detection using the enzyme immunoreaction method.

In a specific embodiment of the present invention, 0.3 mL of zinc sulfate at a concentration of 1 mM to 125 mM was added to 0.2 mL of serum, and the recovery rate of free cortisol according to the concentration of zinc sulfate was measured. Potassium carbonate solution, which is a neutralizing solution, was added in the same equivalent ratio as zinc sulfate. After precipitating the protein and remaining zinc ions, 0.2 mL of sodium acetate solution (pH 5.2) was added to adjust the pH to neutral to use the diluted blood sample for analysis. As a result of analyzing the free cortisol in the diluted blood sample by mass spectrometry, it was found that the relative recovery rate of free cortisol was high when the concentration of zinc sulfate was 1 to 10 mM (Table 1). In addition, after diluting a blood sample in the same manner as above using a 1 to 10 mM zinc sulfate solution, the concentration of free cortisol was measured by the enzyme immunoreaction method. As a result, when the concentration of zinc sulfate was 2 to 5 mM, it was confirmed that the recovery rate of free cortisol was high.

In a preferred embodiment of the present invention, when sodium phosphate solution was used as a neutralization solution, free cortisol recovery of 85% or more was shown in analysis through mass spectrometry, but for optimization of zinc phosphate production, it may be left at room temperature for more than 30 minutes, or it may be stirring for more than 1 minute.

In addition, a previous study (Chinese Patent Publication No. 110702829) analyzing aldosterone by adding a sodium phosphate buffer after precipitating the protein with a zinc sulfate solution, performed additionally proceeding with a solid-phase extraction method based on only pH correction for sample pretreatment. It is a method that is generally used for such the above purpose, and is not used for the purpose of inactivating zinc ions.

In another aspect, the present invention relates to a composition for blood sample dilution solution for measurement of free steroids in blood, characterized by using the method for preparing a diluted blood sample for measuring free steroids in blood according to the present invention, wherein the composition comprises a zinc sulfate solution and an alkaline neutralizing solution.

In another aspect, the present invention relates to a method for measuring free steroids in blood, comprising: (a) precipitating a protein by adding a zinc sulfate (ZnSO₄) solution to a blood sample; (b) neutralizing solution for zinc ion inactivation to the protein-precipitated solution, followed by centrifugation to obtain a supernatant; and (c) measuring the free steroids concentration in the supernatant.

In the present invention, the free steroids concentration in step (c) can be measured using an enzyme immunoreaction method or mass spectrometry method.

In addition, for the specific details of the method for measuring free steroids in blood of the present invention, the above-described “method for preparing a diluted blood sample for measuring free steroids in blood” applies mutatis mutandis.

Hereinafter, the present invention will be described in more detail through examples.

These examples are only for illustrating the present invention, and it will be apparent to those of ordinary skill in the art that the scope of the present invention is not to be construed as being limited by these examples.

### Example 1

### Determination of Free Cortisol Using the Present Blood Sample Dilution Method and Mass Spectrometry

### 1-1: Serum Protein Precipitation

0.3 mL of 1, 2, 5, 10, 20, 50, 100, and 125 mM of zinc sulfate solutions were added, respectively, to a 1.5 mL microtube containing 0.2 mL of human serum, followed by stirring for about seconds.

### 1-2 : Removal of Residual Zinc Ions

0.3 mL of potassium carbonate solution (1, 2, 5, 10, 20, 50, 100, and 125 mM, respectively) of the same molar concentration was added to the above solution, and then stirred for about 10 seconds to precipitate excess zinc ions as zinc carbonate. Then, 0.2 mL of sodium acetate solution (pH 5.2) was added and stirred to neutralize the solution, followed by centrifugation at 8,000 rpm for 5 minutes to obtain a supernatant.

### 1-3: Mass Spectrometry

In order to verify the selective extraction efficiency of free cortisol in the serum sample diluted by the above method, and to measure the concentration, the supernatant obtained in Example 1-2 was analyzed using mass spectrometry (J. Steroid Biochem. Mol. Biol., 198: 105615, 2020).

The concentration of free cortisol in serum samples was compared, and the concentration of free cortisol measured directly in undiluted serum was compared on the basis of 100%

TABLE 1
Extraction recovery of cortisol in diluted serum according
to zinc sulfate concentration (%, mean ± standard deviation)

| zinc sulfate | 1 mM | 2 mM | 5 mM | 10 mM | 20 mM | 50 mM | 100 mM | 125 mM |
|---|---|---|---|---|---|---|---|---|
| recovery | 94.5 ± 4.8 | 96.8 ± 7.7 | 98.3 ± 5.1 | 92.0 ± 3.3 | 78.8 ± 9.8 | 74.5 ± 4.7 | 71.5 ± 5.4 | 70.6 ± 4.0 |
| rate | | | | | | | | |

As a result, when the concentration of zinc sulfate was 1 to 10 mM, the average value of the relative recovery calculated from the diluted serum was high, and the standard deviation was also observed to be within 10% of the reproducible results.

### Example 2

### Measurement of Free Cortisol Using the Present Blood Sample Dilution Method and Enzyme Immunoreaction Method

### 2-1: Serum Protein Precipitation

0.3 mL of 1, 2, 5 and 10 mM of zinc sulfate solutions were added, respectively, to a 1.5 mL microtube containing 0.2 mL of human serum, followed by stirring for about 10 seconds.

### 2-2: Removal of Residual Zinc Ions

0.3 mL of potassium carbonate solution (1, 2, 5 and 10 mM, respectively) of the same molar concentration was added to the above solution, followed by stirring for about seconds to precipitate excess zinc ions as zinc carbonate. Then, 0.2 mL of sodium acetate solution (pH 5.2) was added and stirred to neutralize the solution, followed by centrifugation at 8,000 rpm for minutes to obtain a supernatant.

### 2-3: Enzyme Immune Reaction Method

After dispensing 0.02 mL of the supernatant of Example 2-2 into a 96-well plate provided by the cortisol ELISA kit (abcam, ab108665), 0.2 mL of cortisol-HRP-conjugate (cortisol-HRP conjugate) was added, and reacted at 37° C. for 1 hour. After that, after removing the sample from each well and after washing three times using 0.3 mL of washing solution, 0.1 mL of TMB (3,3′,5,5′-tetramethylbenzidine) solution was added, respectively. After reacting for 15 minutes at room temperature in a dark state, 0.1 mL of reaction stop solution was added and mixed well, and absorbance was measured at 450 nm.

### 2-4: Evaluation of Free Cortisol Concentration

In order to measure cortisol concentration by the enzyme immunoreaction method, standard solutions of 0, 10, 50, 150, and 500 ng/mL were treated in the same manner as the blood sample, and absorbance was measured to prepare a calibration curve. The absolute amount of cortisol detected from the blood sample compared to the standard solution was measured using the following Equation 1 prepared using a four-parameter logistic regression analysis method (4PL), and then the concentration was calculated by dividing it by the volume of blood used.

$$y = \frac{A - D}{1. + {( \frac{x}{c} )}^{B}} + D \qquad Equation 1$$

y: Experimental result value (dependent variable according to the treated sample)
A: Minimum value (blank value when no sample is processed)
B: Slope of the quantitative curve
C: Density at the midpoint of the curve
D: highest value
x: amount of sample treated (independent variable)

TABLE 2
Extraction recovery of cortisol in diluted serum according
to zinc sulfate concentration (%, mean ± standard deviation)

| zinc sulfate | 1 mM | 2 mM | 5 mM | 10 mM |
|---|---|---|---|---|
| recovery rate | 96.9 ± 4.4 | 119.4 ± 2.6 | 146.8 ± 3.7 | 120.1 ± 12.0 |

As a result, as shown in Table 2, the concentration of free cortisol by the enzyme immunoreaction method showed the highest recovery rate when a solution of 2 to 5 mM zinc sulfate was used.

### Example 3

### Cortisol Rrecovery Rates According to Types of Neutralizing Solutions

In the present invention, the recovery rate of free cortisol according to the type of neutralization solution was compared.

First, 0.3 mL of 5 mM concentration of zinc sulfate solution was added to the serum separated from blood in the same manner as in Example 1, followed by stirring for about 10 seconds.

Then, 0.2 M of sodium phosphate buffer solution (pH 7.2) was added instead of potassium carbonate solution as a neutralization solution and stirred for about 1 minute, and then 0.2 mL of sodium acetate solution (pH 5.2) was added and stirred, followed by stirring at 8,000 rpm. The supernatant was obtained by centrifugation for 5 minutes.

Although the analysis through mass spectrometry showed a recovery rate of 85% or more, there may be a disadvantage in that it is necessary to stir for 1 minute or more or leave it at room temperature for 30 minutes or more in order to optimize the production of zinc phosphate.

### Example 4

### Comparison of Free Cortisol Recovery by Sample Type

In the present example, it was attempted to determine whether a whole blood sample could be diluted by the method of the present invention in addition to serum.

The experiment was performed with the blood (whole blood) obtained in Example 1 and the serum separated therefrom, and 0.3 mL of 5 mM concentration of zinc sulfate solution, 0.3 mL of potassium carbonate solution, and 0.2 mL of sodium acetate solution (pH 5.2) were sequentially added to 0.2 mL of each sample, and then centrifuged to obtain a supernatant.

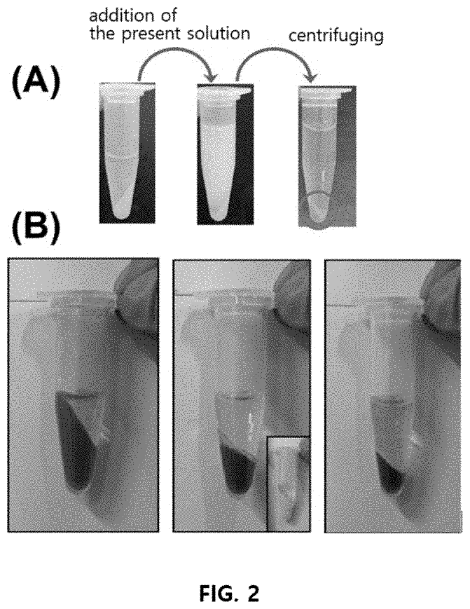

As shown in FIG. 2, the same protein precipitation effect was exhibited in both serum (A) and whole blood (B) samples, and a recovery rate of 94.7 ±3.8% was obtained even in whole blood.

### Example 5

### Confirmation of Applicability of Blood Dilution Method of the Ppresent Invention According to Steroid Type

In order to confirm whether the diluted blood sample preparation method for measuring free steroids in blood of the present invention is applicable to steroids other than cortisol, free testosterone and free cholesterol recovery rates were measured.

The supernatant obtained in Example 1-2 was analyzed using mass spectrometry (J. Steroid Biochem. Mol. Biol., 198: 105615, 2020), and the recovery rates were also compared for testosterone and cholesterol.

As a result, it was confirmed that the recovery rate of free testosterone was 94.3±5.9% and the recovery rate of free cholesterol was 102.5±9.3%, and the diluted solution of the present invention can be selectively separated and detected not only free cortisol, but also other steroid substances present in free form in biological samples.

1. A blood sample dilution solution composition for measurement of free steroids in blood, wherein the composition comprises a zinc sulfate (ZnSO₄) solution and an alkaline neutralizing solution.

2. The blood sample dilution solution composition for measurement of free steroids in blood of claim 1, wherein the steroid is all steroid hormones present in a free form in blood.

3. The blood sample dilution solution composition for measurement of free steroids in blood of claim 2, wherein the steroid hormone is cortisol.

4. The blood sample dilution solution composition for measurement of free steroids in blood of claim 1, wherein the blood is whole blood, serum, or plasma.

5. The blood sample dilution solution composition for measurement of free steroids in blood of claim 1, wherein proteins in the blood are precipitated by the zinc sulfate solution.

6. The blood sample dilution solution composition for measurement of free steroids in blood of claim 1, wherein 1 to 2 mL of 1 to 125 mM zinc sulfate solution is added per 1 mL of the blood sample.

7. The blood sample dilution solution composition for measurement of free steroids in blood of claim 1, wherein the alkaline neutralizing solution has the same molar concentration and addition amount as the zinc sulfate solution.

8. The blood sample dilution solution composition for measurement of free steroids in blood of claim 1, wherein the alkaline neutralizing solution inactivates the remaining zinc ions when the protein is precipitated with the zinc sulfate solution.

9. The blood sample dilution solution composition for measurement of free steroids in blood of claim 1, wherein the alkaline neutralizing solution is anyone selected from the group consisting of sodium hydroxide, potassium hydroxide, sodium carbonate and.

10. The blood sample dilution solution composition for measurement of free steroids in blood of claim 1, wherein the composition further comprises a sodium acetate solution for pH adjustment.

11. The blood sample dilution solution composition for measurement of free steroids in blood of claim 1, wherein the composition is for measuring free steroids in blood by using an enzyme immunoreaction method or mass spectrometry.
